(19) United States
(12) Patent Application Publication
(10) Pub. No.: US 2011/0076261 A1
(43) Pub. Date: Mar. 31, 2011

(54) Antifungal Drug Delivery System

(76) Inventors: Bhiku Patel, Chandler, AZ (US); Craig Woodward, (US); Philip Gordon, (US)

(21) Appl. No.: 12/964,871

(22) Filed: Dec. 10, 2010

Related U.S. Application Data
(63) Continuation-in-part of application No. 12/214,481, filed on Jun. 19, 2008, now abandoned.
(60) Provisional application No. 60/944,873, filed on Jun. 19, 2007.

(51) Int. Cl. A61K 38/48; A61K 31/70; A61K 31/496; A61K 31/192; A61K 31/19; A61K 31/195

(52) U.S. Cl. 424/94.64; 514/28; 514/254.07; 514/383; 514/397; 514/557; 514/23; 514/159; 514/564; 514/588

(57) ABSTRACT

A topical treatment for skin disorders and diseases comprising a combination of at least one antifungal agent and at least one hydroxy acid agent having a pH−pKa value of 0.5 or more formulated into shampoos, creams, lotions, gels, sprays, foams, pads, films, patches, and solutions for treatment of skin disorders and diseases in both humans and animals.

### PRIORITY CLAIM

This Continuation-in-Part application claims the priority of U.S. patent application Ser. No. 12/214,481 filed Jun. 19, 2008, which, in turn claimed the priority of provisional application No. 60/944,873 filed Jun. 19, 2007.

### I. TECHNICAL FIELD

The present invention relates to a topical antifungal agent delivery system and more particularly to a topical antifungal agent delivery system wherein a stable, ionized alpha hydroxy acid or a beta hydroxy acid having a pH minus pKa value of 0.5 or more is used as a non-irritating, effective non-binding carrier which is suited to formulations for use in shampoos, creams, lotions, gels, solutions/serums, sprays, pads, films, patches, and foams for treatment of skin disorders and diseases in both humans and animals.

### II. BACKGROUND OF THE INVENTION AND PRIOR ART

Topical fungal infections have long presented a vexing treatment issue as they often resist treatment. Before resorting to potent oral medications, which can cause a host of problems, including liver damage, physicians prefer to treat fungal infections with a topical medication. While eventually effective in all but the most recalcitrant cases, topical treatments often take months before improvement is seen, which is discouraging to patients, which, in turn, can compound compliance issues. Adding to treatment difficulties is that many topical fungal infections have associated localized hyperkeratotic lesions that adversely affect treatment secondary to poor drug penetration of the lesions as a result of the physical and cellular properties of the hyperkeratotic lesion. Accordingly, removal of thick infected keratin (hyperkeratosis) is useful in creating an environment in which the topical drug can more readily penetrate the fungal lesion. Historically, therefore, hyperkeratotic lesions have been excised prior to topically treating the underlying infection.

Attempts to address the problem via a single topical pharmacologic compound have, until now, been limited by the fact that effective agents for treating the hyperkeratotic lesion and the fungal infection have chemical and/or physical properties that render them instable in the presence of one another. Attempts to address the problem have generally required that the antifungal agent is combined with either a coating agent or a carrying agent.

The present invention advances the art by providing a topical antifungal agent delivery system comprising a stable, ionized alpha hydroxy acid or a beta hydroxy acid having a pH minus pKa value of 0.5 or more with an anti-fungal agent. This unique system enables the skin and the cell wall of fungi to be easily and effectively penetrated while the antifungal agent remains unbound. This, in turn, enables a greater amount of bio-available antifungal agent to reach the infection.

The present invention is unique in that it advances the art by, for the first time in the art, enabling the use of an antifungal agent and an ionized, non-irritating alpha hydroxy acid or a beta hydroxy acid agent in a stable format to create a fungal infection treatment that penetrates easily and is suitable for use in shampoos, creams, lotions, gels, solutions/serums, films, patches, sprays, pads, and foams.

### III. OBJECTS AND ADVANTAGES OF THE PRESENT INVENTION

It is an object of the present invention to create a stable fungal infection treatment delivery system that utilizes ionized hydroxy acid with an antifungal agent.

It is further an object of the present invention to create a stable fungal infection treatment delivery system that effectively treats fungal infections and is also useful for other disorders such as yeast infections.

It is further an object of the present invention to create a fungal infection treatment delivery system in which the hydroxy acid is a beta hydroxy acid or an alpha hydroxy acid.

It is further an object of the present invention to create such a fungal infection treatment that remains stable and is suitable for use as a topical treatment of both human and animal fungal infections and fungal based diseases.

It is further an object of the present invention to create such a fungal infection treatment that is flexible enough that it can be used in multiple formulation such as shampoos, creams, lotions, gels, solutions/serums, films, patches sprays, and foams.

It is further an object of the present invention to create a fungal infection treatment that has optimum pH properties and thus low potential of irritation.

It is further an object of the present invention to create a fungal infection treatment wherein the hydroxy acid is ionized and has a pH−pKa value of 0.5 or greater.

The advantages offered by the present invention include but are not limited to effectively treating skin diseases and disorders in humans and animals through the use of a heretofore unknown stable fungal infection treatment delivery system which eases penetration of hyperkeratotic lesions associated with fungal infections while leaving the antifungal agent unbound. A further advantage of the present invention is that it is effective in treating other infections as well, such as yeast infections. A further advantage of the present invention is increasing efficacy while easing treatment protocols and procedures. A further advantage of the present invention is to decreases the potential for skin irritation and inflammation.

### IV. SUMMARY OF THE INVENTION

The present invention comprises a stable fungal infection treatment delivery system having at least one antifungal agent and at least one ionized beta hydroxy acid compound or at least one ionized alpha hydroxy acid compound or combination thereof for treatment of skin disorders and diseases in both humans and animals. The invention is formulated for topical applications, such as shampoos, creams, lotions, gels, solutions/serums, films, patches, sprays, and foams. The concentration range for the antifungal agent is 0.05% to 10%, and the concentration range for the hydroxy acid agent is 2% to 10% with the acid being ionized and having a pH−pKa value of 0.5 or greater. In the preferred embodiment, the antifungal agent is ciclopirox at a concentration of 0.05% to 3% and the beta hydroxy acid agent is salicylic acid at a concentration of 2% to 6% and a pH−pKa value of approximately 2 (or 99% ionized). The combination can be further combined with surfactants, emulsifiers, solvents, and the like to produce a stable topical dosage form.

There has been outlined, rather broadly, the more important features of the invention in order that the detailed description thereof that follows may be better understood, and in order that the present contribution to the art may be better appreciated. There are, of course, additional features of the invention that will be described hereinafter and that will form the subject matter of the invention.

### V. BRIEF DESCRIPTION OF THE DRAWINGS

FIG. 1 is a photograph of a patient's toe mail at a clinical study baseline.

FIG. 2 is a photograph of the same patient's toenail after one month's use of the inventive formulation in a clinical study.

FIG. 3 is a photograph of the same patient's toenail after three month's use of the inventive formulation in a clinical study.

### VI. DETAILED DESCRIPTION OF THE PREFERRED EMBODIMENT

Before explaining the preferred embodiment of the present invention in detail, it is to be understood that the present invention is not limited in its application to the details of formulations and arrangements of the components set forth in the following description. The present invention is capable of other embodiments and of being practiced and carried out in various ways. Also, it is to be understood that the phraseology and terminology employed herein are for the purpose of description and should not be regarded as limiting. It is also to be understood that where ranges are provided for the various agents and drug examples, they are approximate ranges and are not to be limiting except where noted otherwise.

The present invention contemplates a treatment for fungal infections comprising topical dosage forms for the treatment of human and animal skin disorders and diseases. According to the invention, an antifungal agent and an ionized hydroxy acid are used in combination to create a stable compound effective for the treatment of topical fungal infections as well as other infections such as yeast infections.

Antifungal agents comprise a broad range of therapeutic agents. Non-limiting examples of Synthetic Antifungal Agents are used both topically and systemically include: Pyridone and its derivatives (e.g. Ciclopirox and Ciclopirox Olamine); Azole antifungals, including Imidazoles and its derivatives (e.g. clotrimazole, miconazole, ketoconazole, econazole, terconazole, tioconazole, sertaconazole, butoconazole, oxiconazole, sulconazole, metronidazole, and posoconazole) and Triazoles and its derivatives (e.g. terconazole, itraconazole, fluconazole, etc.); Allylamines and its derivatives (e.g. Terbinafine, neftifine, butenafine, etc.); Tetraene Macrolide (e.g. Nystatin); Polyene Macrolide (e.g. Amphotericin B); Halogenated Phenolic Ether (e.g. Haloprogin); and, Benzoxaborole and its derivatives. The penicillum spp. based antifungal Griseofulvin is also an important and suitable antifungal for use in the invention.

Alpha hydroxy acids and beta hydroxy acids are useful in several skin diseases and disorders and many have hyperkeratolic properties as well. Non-limiting examples of Alpha Hydroxy acids useful in the treatment of skin disorders include lactic acid, mandelic acid, citric acid, glycolic acid, glucuronic acid, and pyruvic acid. Non-limiting examples of beta hydroxy acids useful in the treatment of skin disorders include salicylic acid, Papain, Chymopapain, and Urea.

pKa is a quantitative measure of the strength of an acid in solution. The larger the value of pKa, the smaller the extent of dissociation. A weak acid has a pKa value in the approximate range −2 to 12 in water. Acids with a pKa value of less than about −2 are said to be strong acids. Since pKa can change with temperature, it is customary to measure them, for uniformities sake, at 25° C.

pKa is represented by the following formula:

pKa=−log 10 Ka

pKa values (at 25° C.) for non-limiting examples of acids are:

| | |
|---|---|
| Lactic | 3.85 |
| Mandelic | 3.85 |
| Citric I | 6.5 |
| Citric II | 4.8 |
| Citric III | 6.4 |
| Glycolic | 3.83 |
| Glucoronic | 3.28 |
| Salicylic | 2.97 |
| Urea | 0.10 |
| Papain | 4.10, 8.4 |
| Chymopapain | 6.8 |

pH is a measure of hydrogen ion concentration, acidity or alkalinity, of a solution. Aqueous solutions at 25° C. with a pH less than 7.0 are acidic, while those with a pH greater than seven are basic or alkaline. A pH level of is 7.0 at 25° C. is defined as “neutral.”

pH is represented by the following formula:

$$pH = pKa + log [ \frac{[ Conj Base ]}{[ Acid ]} ]$$

Normally, weak acids will only ionize to a small degree. However, if the pH of the acid is adjusted as compared to its pKa, it ionization level can be altered.

Accordingly, when looking at a particular acid, the percent of ionization of that acid can be determined by the following formula:

$$pH - pKa = + log [ \frac{[ Conj Base ]}{[ Acid ]} ]$$

By definition then, pH−pKa values are as follows:

| Value | Percent ionized |
|---|---|
| −4 | 0.01 |
| −3 | 0.1 |
| −2 | 1.0 |
| −1 | 9.0 |
| 0 | 50 |
| 1 | 91 |
| 2 | 99 |
| 3 | 99.9 |
| 4 | 99.99 |

For non-limiting example, salicylic acid in water has a normal pH of 2.2. This would make the acid approximately 9% ionized (2.2−2.97=−0.77, or approximately −1.0). However, if the pH is raised to 5, the acid is then approximately 99% ionized (5.0−2.97=2.03, or approximately 2).

The Inventors have found that when used in the present invention, acids having a pH−pKa value of 0.5 or greater demonstrate acid ionization levels that move the acids out of the drug or other therapeutic range of effectiveness but do not destroy their carrier properties. This is significant in that the carrier acids enables superior penetration of the drug to the infection but loses its irritation properties. Thus, healing can occur at a faster rate since the anti-fungal agent is delivered to the infection in an efficient manner and tissue is not effected by the acids irritation properties; thus, it does not hinder healing.

The Inventors have also found that the inventive treatment is optimized when beta hydroxy acid is used in a range of about 2% to about 10%, alpha hydroxy acid, when used, in a range of about 5% to about 15% and the antifungal agent is in the range in about 0.05% to about 10%. It should be noted that beta hydroxy acid and alpha hydroxy acid can be used alone or in conjunction or combination with one another. Additionally, more than one antifungal may be used.

In the preferred embodiment, Ciclopirox and salicylic acid are agents of choice. While having different chemical and physical properties from one another, they contain structures that, using the inventive formula and combining process, enable their combination in a stable and effective compound. Though Ciclopirox and salicylic acid are used in the preferred embodiment, this is in no way to be considered limiting in considering the scope of the inventions and the appended claims. Other combinations of antifungals and hydroxy acid agents may also be used in a similar fashion.

Once the antifungal and hydroxy acid are combined according to the description above, they can be further combined with additional components, depending on the intended final use of the product. In a typical formulation, in addition to the antifungal and hydroxy acid combination, any or all of the following may be added without negatively impacting the drugs:
a. surfactants
b. viscosity adjusting agents
c. ph-adjusters
d. stabilizers
e. preservatives
f. moisturizers/humectants
g. fragrance/color

Suitable surfactants can be found in almost any class, including anionic, amphoteric, cationic, non-ionic surfactants. Anionic surfactants have excellent foaming properties, moderate to low irritation potential, and good viscosity building ability. Anionic surfactants include the alkylsulfates, alkylether sulfates, sulfonates, taurates, sulfosuccinates, sacosinates, glutamates, and isothionates.

Anionic synthetic detergents include water-soluble salts, particularly the alkali metal salts, of organic sulfuric reaction products having in their molecular structure an alkyl group containing from about 8 to about 22 carbon atoms and a moiety selected from the group comprising of sulfonic acid and sulfuric acid ester moieties. (Included in the term alkyl is the alkyl portion of higher acyl moieties.) Examples of this group of synthetic detergents are the sodium and potassium alkyl sulfates, especially those obtained by sulfating the higher alcohols (C.sub.8-C.sub.18 carbon atoms) produced by reducing the glycerides of tallow or coconut oil; sodium and potassium alkyl benzene sulfonates, in which the alkyl group contains from about 9 to about 20 carbon atoms in straight-chain or branched-chain configuration; sodium alkyl glyceryl ether sulfonates, especially those ethers of higher alcohols derived from tallow and coconut oil; sodium coconut oil fatty acid monoglyceride sulfonates and sulfates.

Anionic phosphate surfactants are surface active materials having substantial detergent capability in which the anionic solubilizing group connecting hydrophobic moieties is an oxy acid of phosphorus. The more common solubilizing groups, of course, are —SO.sub.4 H and —SO.sub.3 H. Alkyl phosphate esters such as (R—O).sub.2 PO.sub.2 H and ROPO.sub.3 H.sub.2 in which R represents an alkyl chain containing from about 8 to about 20 carbon atoms are useful herein.

These phosphate esters can be modified by including in the molecule from one to about 40 alkylene oxide units, e.g., ethylene oxide units. Formulae for these modified phosphate anionic detergents are ##EQU24## or ##EQU25## in which R represents an alkyl group containing from about 8 to 20 carbon atoms, or an alkylphenyl group in which the alkyl group contains from about 8 to 20 carbon atoms, and M represents a soluble cation such as hydrogen, sodium, potassium, ammonium or substituted ammonium; and in which n is an integer from 1 to about 40.

Another class of suitable anionic organic detergents includes salts of 2-acyloxyalkane-1-sulfonic acids exemplified by the reaction product of fatty acids esterified with isethionic acid and neutralized with sodium hydroxide where, for example, the fatty acids are derived from coconut oil. These salts have the formula ##EQU26## where R.sub.1 is alkyl of about 9 to about 23 carbon atoms (forming with the two carbon atoms an alkane group); R.sub.2 is alkyl of 1 to about 8 carbon atoms; and M is a water-soluble cation.

The water-soluble cation, M, can be, for example, an alkali metal cation (e.g., sodium, potassium, lithium), ammonium or substituted-ammonium cation. Specific examples of substituted ammonium cations include methyl-, dimethyl-, and trimethyl-ammonium cations and quaternary ammonium cations such as tetramethyl-ammonium and dimethyl piperidinium cations and those derived from alkylamines such as ethylamine, diethylamine, triethylamine, mixtures thereof, and the like.

Specific examples of beta-acyloxy-alkane-1-sulfonates, or alternatively 2-acyloxy-alkane-1-sulfonates, useful herein include the sodium salt of 2-acetoxy-tridecane-1-sulfonic acid; the potassium salt of 2-propionyloxy-tetradecane-1-sulfonic acid; the lithium salt of 2-butanoyloxy-tetradecane-1-sulfonic acid; the sodium salt of 2-pentanoyloxy-pentadecane-1-sulfonic acid; the sodium salt of 2-acetoxy-hexadecane-1-sulfonic acid; the potassium salt of 2-octanoyloxy-tetradecane-1-sulfonic acid; the sodium salt of 2-acetoxy-heptadecane-1-sulfonic acid; the lithium salt of 2-acetoxy-octadecane-1-sulfonic acid; the potassium salt of 2-acetoxy-nonadecane-1-sulfonic acid; the sodium salt of 2-acetoxy-uncosane-1-sulfonic acid; the sodium salt of 2-propionyloxy-docosane-1-sulfonic acid; the isomers thereof.

Useful beta-acyloxy-alkane-1-sulfonate salts are the alkali metal salts of beta-acetoxy-alkane-1-sulfonic acids corresponding to the above formula wherein R.sub.1 is an alkyl of about 12 to about 16 carbon atoms, these salts being preferred from the standpoints of their excellent cleaning properties and ready availability.

Another preferred class of anionic detergent compounds herein, both by virtue of superior cleaning properties and low sensitivity to water hardness (Ca++ and Mg++ ions) are the alkylated .alpha.-sulfocarboxylates, containing about 10 to about 23 carbon atoms, and having the formula: ##EQU27## wherein R is C.sub.8 to C.sub.20 alkyl, M is a water-soluble cation as hereinbefore disclosed, preferably sodium ion, and R′ is either short chain length alkyl, e.g., methyl, ethyl, propyl, and butyl or medium chain length alkyl, e.g., hexyl, heptyl, octyl, and nonyl. In the latter case, i.e. the medium chain length esters, the total number of carbon atoms should ideally be in the range 18-20 for optimum performance. These compounds are prepared by the esterification of alpha.-sulfonated carboxylic acids, which are commercially available, using standard techniques. Specific examples of the alkylated.alpha.-sulfocarboxylates preferred for use herein include, short chain length esters (ammonium methyl-.alpha.-sulfopalmitate, triethanolammonium ethyl-.alpha.-sulfostearate, sodium methyl-.alpha.-sulfopalmitate, sodium ethyl-.alpha.-sulfopalmitate, sodium butyl-.alpha.-sulfostearate, potassium methyl-.alpha.-sulfolaurate, and lithium methyl-.alpha.-sulfolaurate, including mixtures); and, medium chain length esters (sodium hexyl-.alpha.-sulphomyristate, potassium octyl-.alpha.-sulpholaurate, ammonium methyl-hexyl-.alpha.-sulpholaurate, and mixtures thereof).

Anionic organic detergents the beta.-alkyloxy alkane sulfonates group are also useful. These compounds have the following formula: ##EQU28## where R.sub.1 is a straight chain alkyl group having from 6 to 20 carbon atoms, R.sub.2 is a lower alkyl group having from 1 (preferred) to 3 carbon atoms, and M is a water-soluble cation as hereinbefore described.

Non-limiting examples of beta.-alkyloxy alkane sulfonates, or alternatively 2-alkyloxy-alkane-1-sulfonates, having low hardness (calcium ion) sensitivity useful herein to provide superior cleaning levels under household washing conditions include, potassium-.beta.-methoxydecanesulfonate, sodium 2-methoxytridecanesulfonate, potassium 2-ethoxytetradecylsulfonate, sodium 2-isopropoxyhexadecylsulfonate, lithium 2-t-butoxytetradecylsulfonate, sodium.beta.-methoxyoctadecylsulfonate, and ammonium .beta.-n-propoxydodecylsulfonate.

Another suitable class of anionic surfactants is the water-soluble salts of the organic, sulfuric acid reaction products of the general formula wherein R.sub.1 is chosen from the group comprising of a straight or branched chain, saturated aliphatic hydrocarbon radical having from 8 to 24, preferably 12 to 18, carbon atoms; and M is a cation. Examples are the salts of an organic sulfuric acid reaction product of a hydrocarbon of the methane series, including iso-, neo-, meso- and n-paraffins, having 8 to 24 carbon atoms, preferably 12 to 18 carbon atoms and a sulfonating agent e.g. SO.sub.3, H.sub.2 SO.sub.4, oleum, obtained according to known sulfonation methods, including bleaching and hydrolysis. Preferred are alkali metal and ammonium sulfonated C.sub.12-18 n-paraffins.

Other useful synthetic anionic detergents are alkyl ether sulfates. These surfactants have the formula RO(C.sub.2 H.sub.4 O).sub.x SO.sub.3 M wherein R is alkyl or alkenyl of about 10 to about 20 carbon atoms, x is 1 to 30, and M is a water-soluble cation as defined hereinbefore. The alkyl ether sulfates useful in the present invention are condensation products of ethylene oxide and monohydric alcohols having about 10 to about 20 carbon atoms. Preferably, R has 14 to 18 carbon atoms. The alcohols can be derived from fats, e.g., coconut oil or tallow, or can be synthetic. Lauryl alcohol and straight chain alcohols derived from tallow are preferred herein. Such alcohols are reacted with 1 to 30, and especially 6, molar proportions of ethylene oxide and the resulting mixture of molecular species, having, for example, an average of 6 moles of ethylene oxide per mole of alcohol, is sulfated and neutralized.

Examples of alkyl ether sulfates of the present invention are sodium coconut alkyl triethylene glycol ether sulfate; lithium tallow alkyl triethylene glycol ether sulfate; and sodium tallow alky hexaoxyethylene sulfate. Especially useful alkyl ether sulphates are those comprising a mixture of individual compounds, said mixture having an average alkyl chain length of from about 12 to 16 carbon atoms and an average degree of ethoxylation of from about 1 to 4 moles of ethylene oxide. Such a mixture also comprises from about 0 to 20% by weight C.sub.12-13 compounds; from 60 to 100% by weight of C.sub.14-15-16 compounds; from about 0 to 20% by weight of C.sub.17-18-19 compounds; from about 3 to 30% by weight of compounds having a degree of ethoxylation of 0; from about 45 to 90% by weight of compounds having a degree of ethoxylation of from 1 to 4; from about 10 to 25% by weight of compounds having a degree of ethoxylation of from 4 to 8; and from about 0.1 to 15% by weight of compounds having a degree of ethoxylation greater than 8.

Additional examples of useful anionic synthetic detergents are those resulting from the reaction product of fatty acids esterified with isethionic acid and neutralized with sodium hydroxide where, for example, the fatty acids are derived from coconut oil; sodium or potassium salts of fatty acid amides of methyl tauride in which the fatty acids, for example, are derived from coconut oil.

Di-anionic detergents compounds, those surfactants containing two anionic functional groups and including the disulfonates, disulfates, or mixtures thereof, where R is an acyclic aliphatic hydrocarbyl group having 15 to 20 carbon atoms and M is a water-solubilizing cation, for example, the C.sub.15 to C.sub.20 disodium 1,2-alkyldisulfates, C.sub.15 to C.sub.20 dipotassium-1,2-alkyldisulfonates or disulfates, disodium 1,9-hexadecyl disulfates, C.sub.15 to C.sub.20 disodium-1,2-alkyldisulfonates, disodium 1,9-stearyldisulfates and 6,10-octadecyldisulfates. The aliphatic portion of the disulfates or disulfonates is generally substantially linear, thereby imparting desirable biodegradable properties to the detergent compound. Water-solubilizing cations include the customary cations known in the detergent art, i.e., the alkali metals, and the ammonium cations, as well as other metals in group HA, IIB, IIIA, IVA and IVB of the Periodic Table except for boron. Preferred water-solubilizing cations are sodium or potassium.

Still other anionic synthetic detergents include the class designated as succinamates. This class includes such surface active agents as disodium N-octadecylsulfosuccinamate; tetrasodium N-(1,2-dicarboxyethyl)-N-octadecylsulfo-succinamate; diamyl ester of sodium sulfosuccinic acid; dihexyl ester of sodium sulfosuccinic acid; dioctyl esters of sodium sulfosuccinic acid.

Other suitable anionic detergents are olefin sulfonates having about 12 to about 24 carbon atoms. The term “olefin sulfonates” is used herein to mean compounds which can be produced by the sulfonation of alpha-olefins by means of uncomplexed sulfur trioxide, followed by neutralization of the acid reaction mixture in conditions such that any sultones which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO.sub.2, chlorinated hydrocarbons, etc., when used in the liquid form, or by air, nitrogen, gaseous SO.sub.2, etc., when used in the gaseous form. When used in the invention, Anionic surfactants should be in a concentration range of about 3% to about 30%.

Amphoteric surfactants are very mild, making them particularly suited for use in personal care and household cleaning products. These surfactants have excellent dermatological properties. They are frequently used in shampoos and other cosmetic products, and also in hand dishwashing liquids because of their high foaming properties. Amphoteric surfactants can be anionic (negatively charged), cationic (positively charged) or non-ionic (no charge) in solution, depending on the acidity or pH of the water. They are compatible with all other classes of surfactants and are soluble and effective in the presence of high concentrations of electrolytes, acids and alkalis. These surfactants may contain two charged groups of different sign. Whereas the positive charge is almost always ammonium, the source of the negative charge may vary (carboxylate, sulphate, sulphonate) and include ordinary alkali metal soaps (e.g. sodium, potassium, ammonium and alkylolamminium salts of higher fatty acids containing from about eight to about 24 carbon atoms and preferably from about 10 to about 20 carbon atoms). Suitable fatty acids can be obtained from natural sources such as, for instance, from plant or animal esters (e.g., palm oil, coconut oil, babassu oil, soybean oil, caster oil, tallow, whale and fish oils, grease, lard, and mixtures thereof). The fatty acids also can be synthetically prepared (e.g., by the oxidation of petroleum, or by hydrogenation of carbon monoxide by the Fischer-Tropsch process). Resin acids are suitable such as rosin and those resin acids in tall oil. Napthenic acids are also suitable. Sodium and potassium soaps can be made by direct saponification of the fats and oils or by the neutralization of the free fatty acids which are prepared in a separate manufacturing process. Particularly useful are the sodium and potassium salts of the mixtures of fatty acids derived from coconut oil and tallow, i.e., sodium or potassium tallow and coconut soap. Betaines are classified generally as amphoteric surfactants. (e.g. cocamidopropyl betaine, sodium cocoamphoacetate)

When used in the invention, amphoteric surfactants should be in a concentration range of about 2% to about 15%.

Cationic Surfactants are quatermary ammonium compounds that acts as a hair conditioners. Examples include cetyltrimethyl ammonium chloride, strearyl dimethyl benzyl ammonium chloride and polyquaterniums. When used in the invention, cationic Surfactants should be in a concentration range of about 0.01% to about 5%.

Nonionic Surfactants are modified linear alcohol ethoxylated compounds and, for example, include glycol fatty esters, sorbitans, tweens, and fatty acid derivatives. When used in the invention, nonionic Surfactants should be in a concentration range of about 2% to about 20%.

In addition to surfactants, viscosity adjusting agents may be added. These agents are used specifically to increase viscosity of product. Polymeric and non-polymeric materials are useful. Examples include acrylate polymers, natural gums-acacia, tregacanth, pectin, etc. When used in the invention, they should be in a concentration range of about 0.1% to about 16%.

pH-adjusters are organic and inorganic acids and bases employed to adjust pH of products to improve physical and chemical stability. Examples include citric acid, lactic acid, sodium hydroxide, ethanolamines, hydrochloric acid, etc. When used in the invention, pH-adjusters would be expected to be in a concentration range of about 0.5% to about 10%, although the final concentration will be in an amount as is necessary for the particular product produced under the invention, and may be outside of this range.

Stabilizers include chelating and anti-oxidant agents such as disodium EDTA, BHT, and BHA, among others. When used in the invention, they should be in a concentration range of about 0.01% to about 2%.

Preservatives serve to preserve products microbiologically during shelf-life of product. Examples include parabens, sorbic acid, germalls, potassium sorbate, and sodium benzoate. When used in the invention, they should be in a concentration range of about 0.1% to about 3%.

Moisturizers, or humectants, may be added to certain formulations. Examples include glycerin, sorbitol, and sodium PCA. When used in the invention, they should be in a concentration range of about 0.5% to about 5%.

Fragrance and/or color may be added if desired. If included, they should be in a concentration range of about 0.01% to about 2%.

Finally, a vehicle is used. Normally this will be water in a range of about 25% to 65%.

It is to be understood that the above discussion represents non-limiting explanations and examples of suitable components. As those skilled in the arts will quickly understand, there are myriad other components, by category or type, that may be used within the scope and spirit of the invention.

### Example 1

### Shampoo #1

An example of the invention is seen below where the inventive formulation is used in a shampoo. The base for each example below contains one or more of the following comprises:
a. vehicle
b. surfactants
c. viscosity adjusting agents
d. ph-adjusters
e. stabilizers
f. preservatives
g. moisturizers/humectants
h. fragrance/color

As can be seen, this shampoo example composition includes salicylic acid 6% and ciclopirox 1%.

| | | |
|---|---|---|
| BETA-HYDROXY ACID | SALICYLIC ACID | 6% |
| ANTIFUNGAL | CICLOPIROX | 1% |
| ANIONIC SURFACTANT | SODIUM LAURETH | 25% |
| | SULFATE | |
| AMPHOTERIC | COCAMIDOPROPYL- | 5% |
| SURFACTANT | BETAINE | |
| CATIONIC SURFACTANT | QUATERNIUM-26 & PG | 0.25% |
| VISCOSITY ADJUSTER | ACRYLATE POLYMER | 10% |
| | XANTAN GUM | 0.50% |
| PH ADJUSTER | TROLAMINE | 6.1% |
| STABILIZER | DISODIUM EDTA | 0.1% |
| MOISTURIZER | GLYCERIN | 1.0% |
| FRAGRANCE | FRAG. CHAMOMILE TEA | 0.1% |
| WATER | WATER | 44.95% |

### Example 2

### Shampoo #2

Here a shampoo composition comprising salicylic acid at 6% and ketoconazole 2%.

| | | |
|---|---|---|
| WATER | DEIONIZED WATER | 43.95% |
| KELTROL CG-T | XANTHAN GUM | 0.50% |
| DISODIUM EDTA | DISODIUM EDTA | 0.10% |
| SALICYLIC ACID USP | SALICYLIC ACID | 6.00% |
| STANDAPOL ES-2 | SODIUM LAURETH SULFATE | 13.00% |
| CARBOPOL AQUA | ACRYLATES COPOLYMER | 10.00% |
| SF-1 | | |
| TEA 99% | TRIETHANOLAMINE | 6.10% |
| STANDAPOL ES-2 | SODIUM LAURETH SULFATE | 12.00% |
| GLUCAM E-10 | METHYL GLUCETH-10 | 1.00% |
| TEGO BETAINE F-50 | COCAMIDOPROPYL BETAINE | 5.00% |
| KETOCONAZOLE USP | KETOCONAZOLE | 2.00% |
| CERAPHYL 65 | QUATERNIUM-26 (AND) | 0.25% |
| | PROPYLENE GLYCOL | |
| FRAGRANCE | CHAMIMILE TEA FRAGRANCE | 0.10% |
| CHAMOMILE TEA | | |

### Example 3

### Solution

Here a solution comprising salicylic acid 6% and Ketoconazole 2%

| | | |
|---|---|---|
| WATER | DEIONIZED WATER | 49.50% |
| NATROSOL 250 HR | HYDROXYETHYLCELLULOSE | 0.40% |
| GLYCERIN USP | GLYCERIN | 2.00% |
| DISODIUM EDTA | DISODIUM EDTA | 0.10% |
| KETOCONAZOLE USP | KETOCONAZOLE | 2.00% |
| ALCOHOL SDA 40, | ALCOHOL | 25.00% |
| 200 PROOF | | |
| SALICYLIC ACID USP | SALICYLIC ACID | 6.00% |
| TEA 99% | TRIETHANOLAMINE | 5.00% |
| ARLASOLVE DMI | DIMETHYL ISOSORBIDE | 5.00% |
| GLUCAM E-10 | METHYL GLUCETH-10 | 5.00% |

### Example 4

### Cream #1

Here a cream comprising ciclopiroxolamine 1%

| | | |
|---|---|---|
| WATER | DEIONIZED WATER | 77.80% |
| KELTROL CG-T | XANTHAN GUM | 0.30% |
| DISODIUM EDTA | DISODIUM EDTA | 0.10% |
| GLYCERIN USP | GLYCERIN | 2.00% |
| OCTYLDODECANOL | OCTYLDODECANOL | 2.00% |
| STEARYL ALCOHOL | STEARYL ALCOHOL | 2.75% |
| SORBITAN | SORBITAN MONOSTEARATE | 1.50% |
| MONOSTEARATE | | |
| MINERAL OIL | MINERAL OIL | 2.00% |
| CETYL ALCOHOL | CETYL ALCOHOL | 2.75% |
| POLYSORBATE 60 | POLYSORBATE 60 | 3.50% |
| LIPOMULSE 165 | GLYCERYL STEARATE (AND) | 3.00% |
| | PEG-100 STEARATE | |
| BENZYL ALCOHOL | BENZYL ALCOHOL | 1.00% |
| CICLOPIROXOLAMINE | CICLOPIROXOLAMINE | 1.00% |
| LACTIC ACID | LACTIC ACID 88% | 0.30% |

### Example 5

### Cream #2

Here a cream comprising ciclopiroxolamine 1% and salicylic acid 6%

| | | |
|---|---|---|
| WATER | DEIONIZED WATER | 66.20% |
| KELTROL CG-T | XANTHAN GUM | 0.30% |
| DISODIUM EDTA | DISODIUM EDTA | 0.10% |
| GLYCERIN USP | GLYCERIN | 2.00% |
| OCTYLDODECANOL | OCTYLDODECANOL | 2.00% |
| STEARYL ALCOHOL | STEARYL ALCOHOL | 2.75% |
| SORBITAN | SORBITAN MONOSTEARATE | 1.50% |
| MONOSTEARATE | | |
| MINERAL OIL | MINERAL OIL | 2.00% |
| CETYL ALCOHOL | CETYL ALCOHOL | 2.75% |
| POLYSORBATE 60 | POLYSORBATE 60 | 3.50% |
| LIPOMULSE 165 | GLYCERYL STEARATE (AND) | 3.00% |
| | PEG-100 STEARATE | |
| BENZYL ALCOHOL | BENZYL ALCOHOL | 1.00% |
| CICLOPIROXOLAMINE | CICLOPIROXOLAMINE | 1.00% |
| LACTIC ACID | LACTIC ACID 88% | 0.30% |
| SALICYLIC ACID USP | SALICYLIC ACID | 6.00% |
| TEA 99% | TRIETHANOLAMINE | 5.60% |

### Example 6

### Cream #3

Here a cream comprising econazole nitrate 2% and salicylic acid 6%

| | | |
|---|---|---|
| WATER | DEIONIZED WATER | 65.20% |
| KELTROL CG-T | XANTHAN GUM | 0.30% |
| DISODIUM EDTA | DISODIUM EDTA | 0.10% |
| GLYCERIN USP | GLYCERIN | 2.00% |
| OCTYLDODECANOL | OCTYLDODECANOL | 2.00% |
| STEARYL ALCOHOL | STEARYL ALCOHOL | 2.75% |
| SORBITAN | SORBITAN MONOSTEARATE | 1.50% |
| MONOSTEARATE | | |
| MINERAL OIL | MINERAL OIL | 2.00% |
| CETYL ALCOHOL | CETYL ALCOHOL | 2.75% |
| POLYSORBATE 60 | POLYSORBATE 60 | 3.50% |
| LIPOMULSE 165 | GLYCERYL STEARATE (AND) | 3.00% |
| | PEG-100 STEARATE | |
| BENZYL ALCOHOL | BENZYL ALCOHOL | 1.00% |
| ECONAZOLE NITRATE | ECONAZOLE NITRATE | 2.00% |
| LACTIC ACID | LACTIC ACID 88% | 0.30% |
| SALICYLIC ACID USP | SALICYLIC ACID | 6.00% |
| TEA 99% | TRIETHANOLAMINE | 5.60% |

### Example 7

### Cream #4

Here a cream comprising ketoconazole 2% and lactic acid 12.0%

| | | |
|---|---|---|
| WATER | DEIONIZED WATER | 50.33% |
| METHOCEL A4M | METHYLCELLULOSE | 0.20% |
| DISODIUM EDTA | DISODIUM EDTA | 0.10% |
| PROPYLENE GLYCOL USP | PROPYLENE GLYCOL | 5.00% |
| KETOCONAZOLE USP | KETOCONAZOLE | 2.00% |
| MINERAL OIL | MINERAL OIL | 5.50% |
| PEG-40 STEARATE | PEG-40 STEARATE | 3.50% |
| CETYL ALCOHOL | CETYL ALCOHOL | 1.00% |
| STEARYL ALCOHOL | STEARYL ALCOHOL | 1.00% |
| LIPO GMS 470 | GLYCERYL STEARATE SE | 6.00% |
| OCTYLDODECANOL | OCTYLDODECANOL | 4.00% |
| LACTIC ACID | AMMONIUM LACTATE | 12.0% |
| | EQUIVILENT TO LACTIC | |
| | ACID | |
| BENZYL ALCOHOL | BENZYL ALCOHOL | 1.00% |

### Example 8

### Foaming Lotion

Here a foaming lotion comprising ciclopiroxolamine 1% and salicylic acid 6%

| | | |
|---|---|---|
| WATER | DEIONIZED WATER | 51.30% |
| NATROSOL 250 HR | HYDROXYETHYLCELLULOSE | 0.10% |
| DISODIUM EDTA | DISODIUM EDTA | 0.10% |
| STANDAPOL ES-2 | SODIUM LAURETH SULFATE | 4.50% |
| GLYCERIN USP | GLYCERIN | 2.00% |
| ALCOHOL SDA 40, | ALCOHOL | 15.00% |
| 200 PROOF | | |
| SALICYLIC ACID USP | SALICYLIC ACID | 6.00% |
| TEA 99% | TRIETHANOLAMINE | 5.00% |
| GLUCAM E-10 | METHYL GLUCETH-10 | 2.50% |
| ARLASOLVE DMI | DIMETHYL ISOSORBIDE | 2.50% |
| CICLOPIROXOLAMINE | CICLOPIROXOLAMINE | 1.00% |
| ALCOHOL SDA 40, | ALCOHOL | 10.00% |
| 200 PROOF | | |

The above examples are for illustrative purpose only. They are intended to provide examples of the versatility of the inventive combinations and should not be considered limiting. Additional combinations for use in cream, lotion, gel, solution/serum, films, patches, foam, spray, and pad dosage forms is also contemplated using the disclosed formulation and preparation standards.

For cream, lotion, gel, solution, foam, spray, film, patches, and pads, the concentration range for the hydroxy acid and antifungal will be:
1. Alpha Hydroxy acid in the range of 5% to 15% (alone or in combination with a beta hydroxy acid)
2. Beta Hydroxy acid in the range of 2% to 10% (alone or in combination with a alpha hydroxy acid)
3. Antifungal agent in the range of 0.1% to 5% (alone or in combination)

The remaining components may include, but are not limited to:

Creams and Lotions:
Vehicle—concentration range: about 25% to about 75%
Oil Phase—fatty acids, alcohols, esters, etc.—concentration range: about 10% to about 50%
Surfactants—anionic, non-ionic, cationic, fatty acids and derivatives-concentration range: about 2% to about 18%
Solvent/Solubilizers—organic alcohols, ethers, esters, salts of fatty acids, glycols, glycerols, etc.—concentration range: about 2% to 3 about 0%
Viscosity adjuster—synthetic polymers and natural gums—concentration range: about 1% to about 15%
Preservatives—concentration range: about 0.05% to about 4%
pH Adjusters—concentration range: about 0.5% to about 15%
Moisturizers—concentration range: about 3% to about 10%
Stabilizers—concentration range: about 0.02% to about 3%
Color and Fragrance—concentration range: about 0.001% to about 2%

Gels—Aqueous, Non-Aqueous, Polymeric, and Non Polymeric- and Solutions:
Solvents—concentration range: about 5% to about 80%
Gelling Agents—concentration range: about 0.1% to about 15%
pH Adjusters—concentration range: about 1% to about 15%
Surfactants—concentration range: about 1% to about 10%
Solubilizer—concentration range: about 0.1% to about 10%
Preservatives—concentration range: about 0.1% to about 5%
Stabilizers—concentration range: about 0.02% to about 3%
Moisturizers—concentration range: about 1% to about 10%

The efficacy of the inventive formula is demonstrated by the following laboratory studies. In study number one, four preparations made having a base corresponding to the shampoo formula listed above (all ingredients with the exception of an antifungal and a hydroxy acid) were produced. The first preparation included Ciclopirox 1% but no hydroxy acid (Preparation 1). The second preparation included Salicylic acid 6% but no antifungal agent (Preparation 2). The third preparation included no active ingredients (Preparation 3). The fourth preparation included Ciclopirox 1% and Salicylic acid 6% (Preparation 4). Each of the preparations was exposed to T. mentagrophytes, var.1 and T. mentagrophytes, var. 2 for one, three, and five minutes. Inoculum level for Var.1 was 1×10⁵ and for Var. 2 was 3.51×10⁵. The results are presented below.

Preparation 1
Ciclopirox 1%, no hydroxy acid

| | |
|---|---|
| Time (min.) | Log Reduction |

T. mentagrophytes, var. 1

| | |
|---|---|
| 1 | 2.13 |
| 3 | 2.09 |
| 5 | 2.15 |

T. mentagrophytes, var. 2

| | |
|---|---|
| 1 | 2.38 |
| 3 | 2.26 |
| 5 | 2.24 |

Preparation 2
Salicylic acid 6%, no antifungal agent

| | |
|---|---|
| Time (min.) | Log Reduction |

T. mentagrophytes, var. 1

| | |
|---|---|
| 1 | 0.14 |
| 3 | 0.26 |
| 5 | 0.18 |

T. mentagrophytes, var. 2

| | |
|---|---|
| 1 | −.38 |
| 3 | 0.07 |
| 5 | 0/15 |

Preparation 3
no active ingredients

| | |
|---|---|
| Time (min.) | Log Reduction |

T. mentagrophytes, var. 1

| | |
|---|---|
| 1 | 0.06 |
| 3 | 0.03 |
| 5 | 0.13 |

T. mentagrophytes, var. 2

| | |
|---|---|
| 1 | −0.27 |
| 3 | −0.19 |
| 5 | −0.27 |

Preparation 4
Ciclopirox 1% and Salicylic acid 6%

| | |
|---|---|
| Time (min.) | Log Reduction |

T. mentagrophytes, var. 1

| | |
|---|---|
| 1 | 5.00 |
| 3 | 5.00 |
| 5 | 5.00 |

T. mentagrophytes, var. 2

| | |
|---|---|
| 1 | 4.55 |
| 3 | 5.55 |
| 5 | 5.55 |

As expected, Preparation 3, no actives, had very little effect on fugal kill rates. Not unexpectedly, Preparation 2, Salicylic acid 6%, no antifungal agent, very little effect, and Preparation 1, Ciclopirox 1%, no hydroxy acid, had some effect on the kill rate, with Preparation 1 (containing an antifungal having significantly higher kill rates than either Preparations 2 or 3. However, Preparation 4, containing the inventive formula Ciclopirox 1% and Salicylic acid 6% had over twice the log reduction kill rate as Preparation 1 which contained only the antifungal agent. Preparation 4 showed no growth for var.1 and 2 at 1, 3, and 5 minutes with 5 log reduction. Preparations 1, 2 and 3 showed growth with lower log reduction.

In study number two, three preparations made having a base corresponding to the cream formula listed above (all ingredients with the exception of an antifungal and a hydroxy acid) were produced. The first preparation included no active ingredients (Preparation 1). The second preparation included Ciclopirox Olamine 1% but no hydroxy acid (Preparation 2). The third preparation included Ciclopirox 1% and Salicylic acid 6% (Preparation 3). Each of the preparations was exposed to C. albicans for five, fifteen, thirty minutes. Inoculum level was 9.75×10⁵. The results are presented below.

Preparation 1
No Active Ingredients
C. Albicans

| Time (min.) | Log Reduction |
|---|---|
| 5 | 0.52 |
| 15 | 0.51 |
| 30 | 0.51 |

Preparation 2
Ciclopirox 1% but no hydroxy acid
C. Albicans

| Time (min.) | Log Reduction |
|---|---|
| 5 | 2.06 |
| 15 | 2.18 |
| 30 | 2.64 |

Preparation 3
Ciclopirox 1% and Salicylic acid 6%
C. Albicans

| Time (min.) | Log Reduction |
|---|---|
| 5 | 5.99 |
| 15 | 5.99 |
| 30 | 5.99 |

As expected, Preparation 1, no actives, had very little effect on fugal kill rates. Not unexpectedly, Preparation 2, antifungal agent but no hydroxy acid had some effect on the kill rate. However, Preparation 4, containing the inventive formula Ciclopirox 1% and Salicylic acid 6% had over twice the log reduction kill rate as Preparation 2. Preparation 3 showed no growth at 5, 15, and 30 minutes with 5.99 log reduction. Preparations 1 and 2 showed growth with lower log reduction.

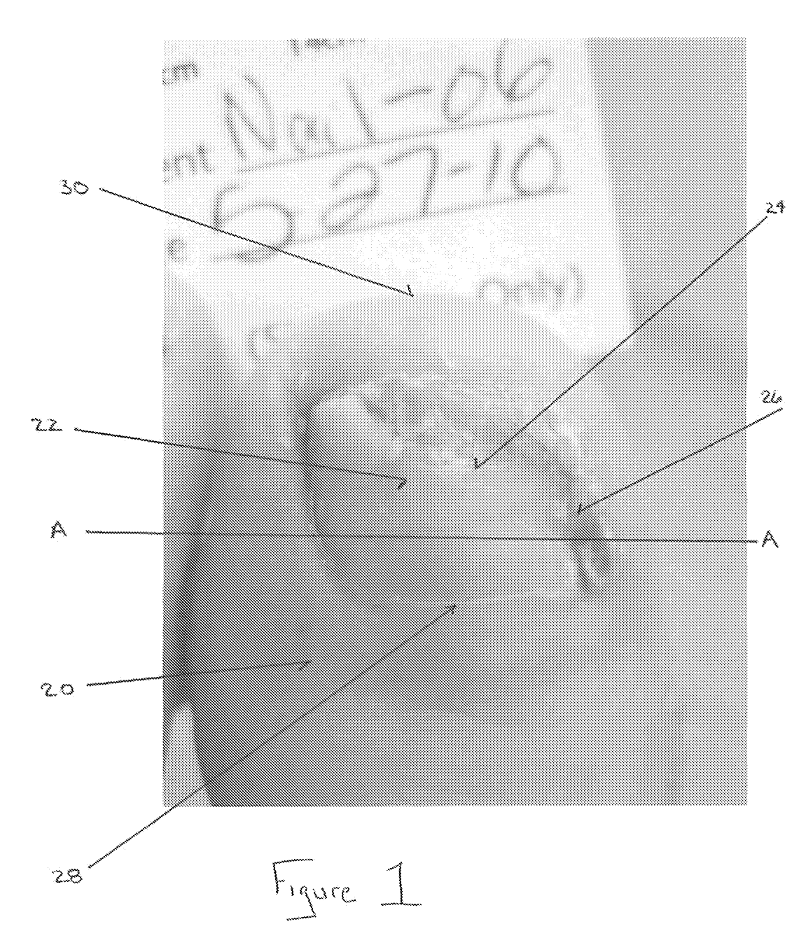

Having achieved significant in vitro results, the Inventors then undertook a human clinical study to prove the effectiveness of the invention. In and IRB approved study, study subjects were screened and confirmed to suffer from onychomycosis in the large toe. Study Subject infections were then photographed at baseline. Turning to FIG. 1, a representative example of an infected toenail at baseline is seen. Large toe 20 has infected nail 22. Onychomycosis infection 24 with scaling 26 can be seen. Nail bed A is seen at approximately 20 percent from the base cuticle 28 to toe distal edge 30. Study subject were then instructed in the use of the inventive formula, a 1% ciclopirox olamine/6% salicylic acid formula was used, which formula was applied twice a day (AM and PM). Study subjects were then instructed to return on a monthly basis for examination, testing, and photographing.

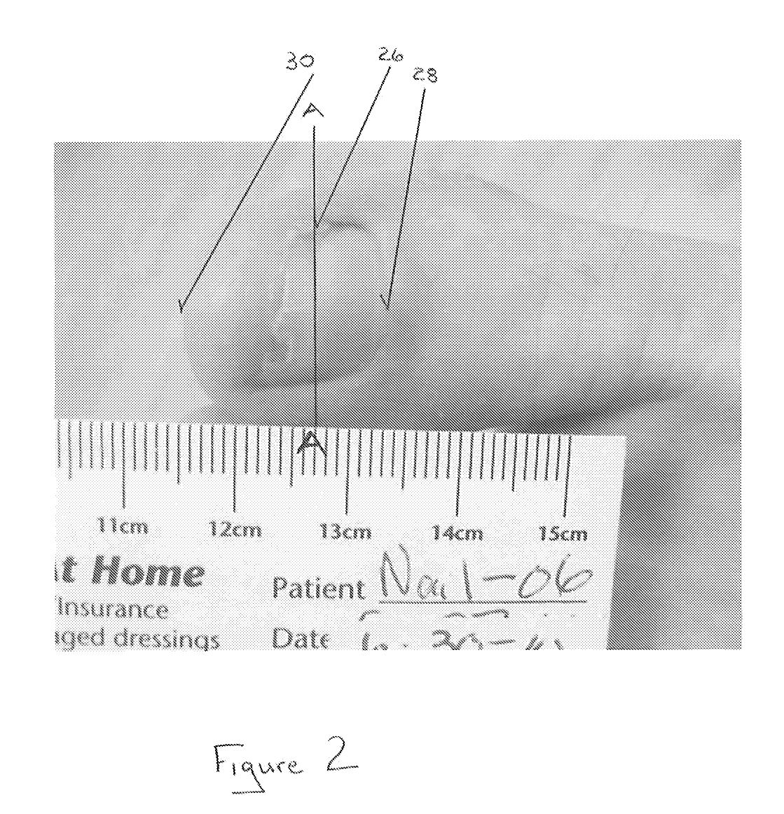

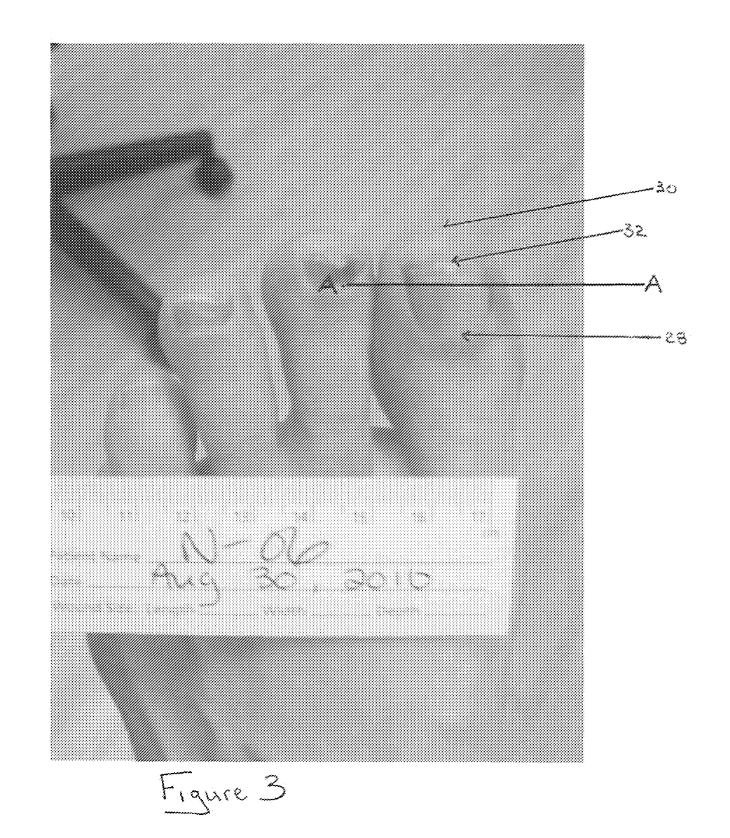

Turning to FIG. 2, study subject returned after one month. As is seen, scaling 26 has improved and nail bed A is now approximately 40% from base cuticle 28 to toe distal edge 30. Further improvement is seen in FIG. 3, after month three, scaling is cleared and nail bed A is approximately 60% from base cuticle to toe distal edge 30 and almost clear of infection to nail distal edge 32.

As theoretically expected, in use Study subjects reported no irritation with the treatment regimen.

It is to be understood, however, that even though numerous characteristics and advantages of the preferred and alternative embodiments have been set forth in the foregoing description, together with details of the structure and function of the embodiments, the disclosure is illustrative only, and changes may be made in detail within the principles of the invention to the full extent indicated by the broad general meaning of the terms in which the appended claims are expressed.

We claim:

1. A composition for the treatment of skin disorders and diseases comprising a delivery system further comprising at least one antifungal agent and at least one hydroxy acid wherein the at least one hydroxy acid has a pH−pKa value of 0.5 or more.

2. The composition of claim 1 wherein the at least one antifungal agent is selected from the group consisting of Ciclopirox, Ciclopirox Olamine, clotrimazole, miconazole, ketoconazole, econazole, terconazole, tioconazole, sertaconazole, butoconazole, oxiconazole, sulconazole, metronidazole, posoconazole, terconazole, itraconazole, fluconazole, Terbinafine, neftifine, butenafine, Nystatin, Amphotericin B, Haloprogin, Griseofulvin, and a Benzoxaborole.

3. The composition of claim 1 wherein the at least one hydroxy acid is selected from the group consisting of lactic acid, mandelic acid, citric acid, glycolic acid, glucuronic acid, pyruvic acid, salicylic acid, papain, chymopapain, and Urea.

4. The composition of claim 1 where the at least one antifungal agent is in a range of about 0.05% to about 10%.

5. The composition of claim 1 where the at least one hydroxy acid is a beta hydroxy acid in a range of about 2% to about 10% or an alpha hydroxy acid of about 5% to about 15%.

6. The composition of claim 1 where the at least one antifungal agent is Ciclopirox.

7. The composition of claim 1 where the at least one antifungal agent is econazole.

8. The composition of claim 1 where the at least one antifungal agent is ketoconazole.

9. The composition of claim 1 where the at least one hydroxy acid is salicylic acid.

10. The composition of claim 1 where the at least one hydroxy acid is lactic acid.

11. The composition of claim 1 further including base ingredients selected of one or more from the group consisting of surfactants, viscosity adjusting agents, ph-adjusters, stabilizers, preservatives, moisturizers/humectants, fragrance, and color.

12. The surfactants of claim 8 selected from the family groups of surfactants consisting of anionic, amphoteric, cationic, and non-ionic.

13. The composition of claim 1 further formulated into any of a shampoo, cream, lotion, gel, solution, serum, spray, pad, film, patch, or foam.

14. A composition for the treatment of skin disorders and diseases comprising a delivery system further comprising at least one antifungal agent and salicylic acid wherein the salicylic acid has a pH−pKa value of 0.5 or more.

15. The composition of claim 14 wherein the at least one antifungal agent is selected from the group consisting of Ciclopirox, Ciclopirox Olamine, clotrimazole, miconazole, ketoconazole, econazole, terconazole, tioconazole, sertaconazole, butoconazole, oxiconazole, sulconazole, metronidazole, posoconazole, terconazole, itraconazole, fluconazole, Terbinafine, neftifine, butenafine, Nystatin, Amphotericin B, Haloprogin, Griseofulvin, and a Benzoxaborole.

16. The composition of claim 14 where the at least one antifungal agent is in a range of about 0.05% to about 10%.

17. The composition of claim 14 where the salicylic acid in a range of about 2% to about 10%.

18. The composition of claim 14 where the at least one antifungal agent is Ciclopirox.

19. The composition of claim 14 where the at least one antifungal agent is econazole.

20. The composition of claim 14 where the at least one antifungal agent is ketoconazole.

21. The composition of claim 14 further including base ingredients selected from the group consisting of surfactants, viscosity adjusting agents, ph-adjusters, stabilizers, preservatives, moisturizers/humectants, fragrance, and color.

22. The surfactants of claim 14 selected from the family groups of surfactants consisting of anionic, amphoteric, cationic, and non-ionic.

23. The composition of claim 14 further formulated into any of a shampoo, cream, lotion, gel, solution, serum, spray, pad, film, patch, or foam.

24. A composition for the treatment of skin disorders and diseases comprising in combination at least one antifungal agent and lactic acid.

25. The composition of claim 24 wherein the at least one antifungal agent is selected from the group consisting of Ciclopirox, Ciclopirox Olamine, clotrimazole, miconazole, ketoconazole, econazole, terconazole, tioconazole, sertaconazole, butoconazole, oxiconazole, sulconazole, metronidazole, posoconazole, terconazole, itraconazole, fluconazole, Terbinafine, neftifine, butenafine, Nystatin, Amphotericin B, Haloprogin, Griseofulvin, and a Benzoxaborole.

26. The composition of claim 24 where the at least one antifungal agent is in a range of about 0.05% to about 10%.

27. The composition of claim 24 where the lactic acid in a range of about 5% to about 15%.

28. The composition of claim 24 where the at least one antifungal agent is Ciclopirox.

29. The composition of claim 24 where the at least one antifungal agent is econazole.

30. The composition of claim 24 where the at least one antifungal agent is ketoconazole.

31. The composition of claim 24 further including base ingredients selected from the group consisting of surfactants, emulsifiers, viscosity adjusting agents, ph-adjusters, stabilizers, preservatives, moisturizers/humectants, fragrance, and color.

32. The surfactants of claim 24 selected from the family groups of surfactants consisting of anionic, amphoteric, cationic, and non-ionic.

33. The composition of claim 24 further formulated into any of a shampoo, cream, lotion, gel, solution, serum, spray, pad, film, patch, or foam.

34. A composition for the treatment of skin disorders and diseases comprising a delivery system further comprising at least one antifungal agent, at least one hydroxy acid having a pH−pKa value of 0.5 or more, and at least one surfactant.

35. The composition of claim 34 further including base ingredients selected from the group consisting of a viscosity adjusting agent, a ph-adjuster, a stabilizer, a preservative, a moisturizers/humectants, a fragrance, and a color.

36. The composition of claim 34 wherein the at least one antifungal agent is selected from the group consisting of Ciclopirox, Ciclopirox Olamine, clotrimazole, miconazole, ketoconazole, econazole, terconazole, tioconazole, sertaconazole, butoconazole, oxiconazole, sulconazole, metronidazole, posoconazole, terconazole, itraconazole, fluconazole, Terbinafine, neftifine, butenafine, Nystatin, Amphotericin B, Haloprogin, Griseofulvin, and a Benzoxaborole.

37. The composition of claim 34 wherein the at least one hydroxy acid is selected from the group consisting of lactic acid, mandelic acid, citric acid, glycolic acid, glucuronic acid, pyruvic acid, salicylic acid, Papain, Chymopapain, and Urea.

38. The composition of claim 34 where the at least one antifungal agent is in a range of about 0.05% to about 10%.

39. The composition of claim 34 where the at least one hydroxy acid is a beta hydroxy acid in a range of about 2% to about 10% or an alpha hydroxy acid of about 5% to about 15%.

40. The composition of claim 34 where the at least one antifungal agent is Ciclopirox.

41. The composition of claim 34 where the at least one hydroxy acid is salicylic acid.

42. The composition of claim 34 further including base ingredients selected from the group consisting of surfactants, emulsifiers, viscosity adjusting agents, ph-adjusters, stabilizers, preservatives, moisturizers/humectants, fragrance, and color.

43. The surfactants of claim 34 selected from the family groups of surfactants consisting of anionic, amphoteric, cationic, and non-ionic.

44. The composition of claim 34 further formulated into any of a shampoo, cream, lotion, gel, solution, serum, spray, pad, film, patch, or foam.
